(19) United States
(12) Patent Application Publication
(10) Pub. No.: US 2025/0324977 A1
(43) Pub. Date: Oct. 23, 2025

(54) ENGINEERED BACTERIA FOR ENHANCED CROP PRODUCTION

(71) Applicant: The Regents of the University of California, Oakland, CA (US)

(72) Inventors: Yasuo Yoshikuni, Orinda, CA (US); Bing Wang, Berkeley, CA (US)

(21) Appl. No.: 18/871,025

(22) PCT Filed: Jun. 1, 2023
(86) PCT No.: PCT/US2023/024188

§ 371 Date: Dec. 2, 2024

Related U.S. Application Data
(60) Provisional application No. 63/348,476, filed on Jun. 2, 2022.

(51) Int. Cl. A01N 63/27; A01P 21/00; C12N 9/02; C12N 9/10; C12N 9/78; C12N 9/88; C12N 15/52; C12N 15/78

(52) U.S. Cl. CPC A01N 63/27; A01P 21/00; C12N 9/0008; C12N 9/1085; C12N 9/1096; C12N 9/78; C12N 9/88; C12N 15/52; C12N 15/78; C12Y 102/03007; C12Y 205/01054; C12Y 206/01027; C12Y 305/99007; C12Y 401/01074; C12Y 401/03027

(57) ABSTRACT

The present invention provides for a plant-growth-promoting rhizobacterium (PGPR) genetically modified such that the PGPR is capable of colonizing the root of a plurality of plant species, such as a plurality of crop plant species, wherein the genetically modified PGPR is enhanced in the capability to colonize the root of a plurality of plant species when compared to a wild-type or unmodified PGPR.

### CROSS-REFERENCE TO RELATED APPLICATIONS

This application claims priority to U.S. Provisional Patent Application Ser. No. 63/348,476, filed Jun. 2, 2022, which is incorporated by reference in its entirety.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of enhancing crop production.

### BACKGROUND OF THE INVENTION

The world is gradually awakening to the urgency of addressing climate change. The sustainable development scenario (SDS) of the International Energy Agency (IEA) requires reducing annual global CO₂ emissions from 2019's 35.8 billion tons to less than 10 billion tons by 2050. This reduction would be needed to meet the Paris climate change conference (COP21) objectives for keeping temperature rise this century below 2.0° C. However, we lag far behind these goals. Meanwhile, the United Nations estimates that global population will grow to almost 10 billion by 2050 (United Nations, 2019). This means that demands for crops for food and energy will increase dramatically. However, agriculture, forestry, and other land use already contribute 24% of global greenhouse gas emissions. Agricultural production must increase in a sustainable way, without increased reliance on modern technology that has a high CO₂ footprint (e.g., chemical fertilizers) or that raises environmental or health concerns (e.g., chemical pesticides). Further exacerbating the problem, climate change will likely lower crop yields because of increased temperature stress and extreme weather events such as droughts (Zandalinas et al., 2021). In addition, soil salinity will be increased by saltwater intrusion caused by rising sea levels and by depletion of groundwater (Chen and Mueller, 2018). Climate change will also alter the geographic distribution of crop diseases by introducing pathogens to new areas (Anderson et al., 2004). The world needs sustainable ways to increase agricultural production despite the ever-increasing adverse impacts of climate change on agriculture.

Plant roots are associated with tens of thousands of different bacterial species (Müller et al., 2016). Plant roots provide sustenance for these bacteria by secreting 20-40% of the carbon fixed through photosynthesis to the rhizosphere (Ryan et al., 2001). In turn, some bacteria known as plant-growth-promoting rhizobacteria (PGPRs) can benefit plants, acting as bio-fertilizers, bio-stimulants, and bio-pesticides (Finkel et al., 2017). These PGPRs suggest strategies for reducing our reliance on chemical fertilizers and pesticides. Many commercial PGPR products are currently available. The global market value of these products is nearly USD 7 billion and is growing rapidly, primarily driven by organic farmers (Timmusk et al., 2017). However, PGPR effects vary depending on crop species and environment, and the large-scale utility of PGPRs is still limited (Backer et al., 2018). Current understanding is that externally added PGPRs are likely excluded by native microbiomes (Ahkami et al., 2017), which maintain their dominance resiliently in the face of PGPR addition.

### SUMMARY OF THE INVENTION

The present invention provides for a plant-growth-promoting rhizobacterium (PGPR) genetically modified such that the PGPR is capable of colonizing the root of a plurality of plant species, such as a plurality of crop plant species, wherein the genetically modified PGPR is enhanced in the capability to colonize the root of a plurality of plant species when compared to a wild-type or unmodified PGPR. A rhizobacterium is any root-associated bacterium, such as a bacterium that in nature colonizes plant roots, or is found in proximity with a plant root. The modified PGPR is enhanced or increased in its capability to promote, enhance or increase one or more of its plant-growth-promoting (PGP) traits, property, or activity, when compared to the unmodified PGPR.

In some embodiments, the PGPR is genetically modified in a method described herein. In some embodiments, the PGPR is genetically modified such that the PGPR modulates, such as increase or decrease, the effects of one or more plant hormones, such as an auxin and/or ethylene. In some embodiments, the auxin is indole-3-acetic acid (IAA). In some embodiments, the unmodified PGPR naturally does not, or produces less, auxin or ethylene when compared to the genetically modified PGPR. In some embodiments, the PGPR is genetically modified to express enzyme(s) capable of producing an auxin in the PGPR. In some embodiments, the enzyme(s) are heterologous to the PGPR. In some embodiments, the enzyme(s) are endogenous to the PGPR but the endogenous enzyme(s) produce less of the auxin as compared to the genetically modified PGPR.

The present invention provides for a method of genetically modifying a PGPR, comprising introducing and/or deleting one or more gene(s) in the PGPR. In some embodiments, the gene is heterologous to PGPR, and/or the gene is a homologous gene thereof.

The present invention provides for a method of enhancing growth of a plant, comprising introducing a genetically modified PGPR of the present invention into the soil, or vicinity of a plant or plant root.

In some embodiments, the PGPR is modified to produce an auxin or ethylene. In some embodiments, the PGPR is modified to express one or more, or all, of the following enzymes: aminotransferase, IPyA decarboxylase (IPDC), and/or indole acetaldehyde dehydrogenase (IAAID). In some embodiments, the PGPR is naturally capable of producing tryptophan. In some embodiments, the PGPR is naturally not capable of producing tryptophan, wherein the PGPR is modified to express 3-Deoxy-D-arabinoheptulosonate 7-phosphate (DAHP) synthase and/or anthranilate synthetase so that the PGPR can produce tryptophan. In some embodiments, the PGPR is modified to express or increase expression of 1-aminocyclopropane-1-carboxylate (ACC) oxidase, and/or decrease, or knock out, expression of ACC deaminase. In some embodiments, the PGPR is modified to decrease, or knock out, expression of an endogenous ACC deaminase.

In some embodiments, the PGPR is modified to comprise one or more nucleic acids encoding the enzyme(s) described herein, which are operatively linked to one or more promoters capable of expression of the enzyme(s) in the PGPR. In some embodiments, the nucleic acid(s) is a heterogenous DNA. In some embodiments, the nucleic acid resides on a vector or expression vector which stably resides in the cytoplasm of the PGPR, or the nucleic acid is stably integrated into the genome of the PGPR.

The present invention provides for a method of constructing the PGPR of the present invention, the method comprising introducing a nucleic acid encoding one or more enzyme(s) described herein into the unmodified PGPR, such that the modified PGPR is capable of expressing the one or more enzyme(s).

The present invention provides for a method of enhancing growth of a plant, the method comprising introducing or applying or contacting the PGPR of the present invention to or with a soil, wherein a plant resides, or a plant, such that the PGPR colonizes the soil so that growth of the plant is enhanced. In some embodiments, the growth of the plant is enhanced in that the stem, shoot, or root of the plant has more growth, such as in weight and/or length, compared to a similar treatment with an unmodified PGPR. In some embodiments, the enhanced growth is to a degree described herein or within a range of any two values described herein. In some embodiments, the growth of the plant, or a plant part, such as the stem, shoot, or root, such as weight and/or length, is further enhanced at least about 10%, 20%, 30%, 40%, 50%, 60%, 70%, 80%, 90%, or 100%, or by a value in a range within any two preceding values, when compared to the enhancement by an unmodified PGPR.

Using synthetic biology to engineer PGPRs that can robustly colonize the roots of diverse crop species may be an ideal way to tackle this global challenge. In some embodiments, the PGPR is an engineered Pseudomonas simiae WCS417, which possesses several interesting plant-growth-promoting (PGP) traits natively.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Domestication of six Arabidopsis roots colonizing bacteria with CRAGE-DUET. a, The structure of CRAGE-DUET DNA cassette. b, Species tree. The green-colored nodes belong to alpha-proteobacteria and the red for beta-proteobacteria, and the blue for gamma-proteobacteria. The nearby heatmap represents resistance profiles for each strain against APR (apramycin), Kan (kanamycin), CHL (chloramphenicol), AMP (ampicillin), GEN (gentamicin), TET (Tetracycline), and SPT (spectinomycin). The resistance profiles are determined by Venus fluorescence intensities measured on plate reader. The incubation for each strain was induced by containing 1 nM, 0.1 mM, 0.01 mM, and 0.001 mM IPTG, respectively. The fluorescence intensity was measured at λExc/λEm≈515/528 nm. d, Stress response of roots treated with engineered P. simiae WCS417.

FIG. 2. High-throughput strains engineering for identifying high-performing IAA pathway assembly or ACC deaminase gene. a, Combinatorial assembly of IAA pathway using Golden gate assembly approach. IAA version1 (IAAv1) contains enzymes AAT, IPDC, and IaaId, and IAAv2 with IPDC, DAHP, and AS enzyme. Each enzyme has 5 or 6 or 7 encoding candidate genes for the assemblies. Finally, 462 IAA pathway variants were built. b, High-throughput conjugations for integrating IAA pathway and ACC deaminase gene (acdS) and IPTG inducible LacUV5 driven T7RNAP into CRAGE-Duet inserted P. simiae WCS417. c, Clustering analysis of IAA product of engineered IAA strains measured with Salkowski colorimetric reaction. d, LC-MS quantification of IAA product and ACC content in the incubations of selected IAA and acdS strains.

FIG. 3. Identification of constitutive LacUV promoter mutants of different strengths. a, LacUV mutants library construction. Oligo DNA of LacUV sequence that was deleted with lacI operator motif and containing degenerate nucleotides replacement between −35 and −10 was cloned into CRAGE-Duet inserted P. simiae WCS417 strain carrying a GFP reporter gene. The nucleotide sequence depicted is SEQ ID NO:1. b, Library-quality analysis. 4 M unique mutant sequences were identified by miSeq. Around 70% of mutant sequences are a single copy, and 20% have an average of 28 copies. c, GFP fluorescent intensity for strains carrying different constitutive LacUV mutants. d, Fluorescent imaging of GFP strains with strong and weak constitutive LacUV mutant.

FIG. 4. Arabidopsis roots growth after inoculation of engineered IAA and acdS P. simiae WCS417 strain. a. Lengths of primary roots and lateral roots one week after nine different treatments containing wild-type (wt), weak acdS (w.acdS), strong acdS (S.acdS), weak IAA (w.IAA), the combination of w.acdS plus w.IAA, the combination of S.acdS plus w.IAA, strong IAA (s. IAA), the combination of w.acdS plus s.IAA, and the combination of s.acdS plus s.IAA P. simiae WCS417 strain. Each treatment has 24 seedlings. The inoculation was performed by spotting 1 μL of 0.02 OD600 strains on the root tip of seedlings. b, Lengths of primary roots two weeks after inoculations. C, Representative images for two weeks seedlings inoculated with wt WCS417 and the combination of s.acdS plus s.IAA. d, Pot assay. Two weeks post inoculation seedlings were transferred into soil pots. The pots were divided into two groups, one growing under a normal condition and the other under a drought condition by holding on to water for over two weeks. For statistical significance, the double asterisks mean a level of less than or equal to 1% and one asterisk for less than or equal to 5%. Except for specific annotations, the pairwise comparisons were performed between wt WCS417 and engineered IAA/acdS strains or their combinations.

FIG. 5. Microscopic imaging of the interactions of IAA and acdS strains with Arabidopsis roots.

FIG. 6. Conjugations for inserting CRAGE-DUET into the genomes of six non-model bacteria. a, Emerging colonies on selective Kna plates following the conjugations of donor strain WM3064 containing CRAGE-DUET with six bacteria, including Pseudomonas simiae sp. WCS417, Ochrobactrum sp. 370MFChir3.1, Rhizobium sp. 2MFCol3.1, Agrobacterium sp. 33MFTal.1, Ralstonia sp. UNC404CL21Col and Pseudomonas sp. 397MF. b, Counter selection. After one round of conjugation to investigate Venus gene into CRAGE-DUET inserted strains, emerging colonies were made two copies, one spotted on selective Kna and one on Apr plates. c, Colony PCR. The PCRS were for confirming the integration of IPTG inducible Lac T7RNAP into the above Venus integrated strains following the other round of conjugations. For each strain, the upper panel indicates the integration of T7RNAP into the Venus carrying CRAGE-DUET strain, and the lower panel suggests the absence of donor plasmid carrying Lac T7 RNAP.

FIG. 7. Pathways for tryptophan and IAA and ethylene biosynthesis. a, IAA synthesis via the IPy route. b, Shikimate pathway. c, Ethylene synthesis.

FIG. 8. Vectors map. a, The plasmid carrying CRAGE-DUET cassette. b, The plasmid for cloning pathway/gene through BsaI digestion and ligation c, The plasmid for cloning desired promoter to drive T7RNAP through BsaI digestion and ligation.

FIG. 9. Salkowski colorimetric assay for the measure of IAA products. a, Histogram chart showing the distributions of IAA yield for 210 IAAv1 and 252 IAAv2 strains. b, Ranking candidate genes for each enzyme based on IAA yield for IAAv1 strains. c, for IAAv2 strains.

FIG. 10. LC-MSMS analysis of IAA and acdS strains incubations. a, IAA yield. The MSMS spectrum for compound IAA was observed at 176.0708 in positive ESI mode that matches its theoretical mass. Extracted ion chromatograms of IAA were for ten selected IAA strains. b, acdS activity. The MSMS spectrum for compound ACC was observed at 102.0551 in positive ESI mode that matches its theoretical mass. Extracted ion chromatograms of ACC were for six selected acdS strains.

FIG. 11. WebLogo showing conserved nucleotides for promoter mutants of high, medium, and low strength—created at WebLogo (website for: weblogo.threeplusone.com/). The conservation evaluation is shown by the height of the stack of letters. The nucleotide sequence depicted (CTTTA TGCTT CCGGC TCG) is SEQ ID NO:2. In some embodiments, the promoter is a strong promoter comprising SEQ ID NO: 2, wherein one or more of the following nucleotides are at the indicated position: A or T at 1, A or G at 3, C or A at 5, T or G at 11, T at 16, and T or G at 18. In some embodiments, the promoter is a medium promoter comprising SEQ ID NO:2, wherein one or more of the following nucleotides are at the indicated position: T at 4, T at 6, T at 9, and G or T at 18. In some embodiments, the promoter is a weak promoter comprising SEQ ID NO:2, wherein one or more of the following nucleotides are at the indicated position: A or T at 4, a or C at 8, and A or C at 15.

FIG. 12. Phenotype of Arabidopsis triple mutant wei8 tar1 tar2 inoculated with wildtype or weak IAA P. simiae WCS417 strain. First, each seed of the triple mutant was inoculated with 1 μL of OD600 0.02 suspension of wt or the combination of strong IAA and strong acdS strain. The inoculated seeds were vertically incubated in plant growth chamber for three weeks. Afterward, the seedlings were transferred into soil pots. The pots were incubated under normal conditions with 16/8 h light/dark cycle.

FIG. 13. Phenotypes of Arabidopsis roots inoculated with different levels of bacteria. 1 μL of wt or the combination of strong IAA and strong acdS strain was spotted on the tip of individual on-week-old Arabidopsis Col-0 seedling. The inoculants concentration is at the level of OD600 of 0.2, 0.02, 0.002, and 0.0002. The inoculated were vertically incubated in plant growth chamber for two weeks.

FIG. 14. Phenotypes of Arabidopsis roots treated with nine different inoculations. The nine treatments contain wild-type (wt), weak acdS (w.acdS), strong acdS (S.acdS), weak IAA (w.IAA), the combination of w.acdS plus w.IAA, the combination of S.acdS plus w.IAA, strong IAA (s. IAA), the combination of w.acdS plus s.IAA, and the combination of s.acdS plus s.IAA P. simiae WCS417 strain. 1 μL of the suspension of OD600 0.02 for each treatment was spotted on the tip of individual on-week-old Arabidopsis Col-0 seedling. The inoculated were vertically incubated in plant growth chamber for two weeks.

### DETAILED DESCRIPTION OF THE INVENTION

Before the invention is described in detail, it is to be understood that, unless otherwise indicated, this invention is not limited to particular sequences, expression vectors, enzymes, PGPRs, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “PGPR” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given PGPR; (b) the sequence may be naturally found in a given PGPR, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given PGPR; or (b) the structure or molecule may be naturally found in a given PGPR, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a PGPR, wherein the expression vector contains a nucleic acid sequence coding for an enzyme that is not normally found in a PGPR. With reference to the PGPR's genome, then, the nucleic acid sequence that codes for the enzyme is heterologous.

The terms “expression vector” or “vector” refer to a compound and/or composition that transduces, transforms, or infects a PGPR, thereby causing the cell to express nucleic acids and/or proteins other than those native to the cell, or in a manner not native to the cell. An “expression vector” contains a sequence of nucleic acids (ordinarily RNA or DNA) to be expressed by the PGPR. Optionally, the expression vector also comprises materials to aid in achieving entry of the nucleic acid into the PGPR, such as a virus, liposome, protein coating, or the like. The expression vectors contemplated for use in the present invention include those into which a nucleic acid sequence can be inserted, along with any preferred or required operational elements. Further, the expression vector must be one that can be transferred into a PGPR and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a PGPR or cell. Only when the sequence of nucleic acids becomes stably replicated by the cell does the PGPR or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a PGPR, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

### Enzymes, and Nucleic Acids Encoding Thereof

A homologous gene or enzyme is a gene or enzyme that has a polypeptide sequence that is at least 70%, 75%, 80%, 85%, 90%, 95% or 99% identical to any one of the genes or enzymes described in this specification or in an incorporated reference. The homologous genes or enzyme retains amino acids residues that are recognized as conserved for the genes or enzyme. The homologous genes or enzyme may have non-conserved amino acid residues replaced or found to be of a different amino acid, or amino acid(s) inserted or deleted, but which does not affect or has insignificant effect on the enzymatic activity of the homologous genes or enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject genes or enzymes. The nucleic acid of the subject genes or enzymes is operably linked to promoters and optionally control sequences such that the subject genes or enzymes are expressed in a PGPR cultured under suitable conditions. The promoters and control sequences are specific for each PGPR species. In some embodiments, expression vectors comprise the nucleic acid constructs. Methods for designing and making nucleic acid constructs and expression vectors are well known to those skilled in the art. In some embodiments, the promoter is one described herein, such as in the description for FIG. 11.

Sequences of nucleic acids encoding the subject genes or enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject genes or enzyme can be incorporated into an expression vector. Incorporation of the individual nucleic acid sequences may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a PGPR simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBRIMCS-3, pUR, pEX, pMR100, pCR4, pBAD24, pUC19; bacteriophages, such as M13 phage and λ phage. Of course, such expression vectors may only be suitable for particular PGPRs. One of ordinary skill in the art, however, can readily determine through routine experimentation whether any particular expression vector is suited for any given PGPR. For example, the expression vector can be introduced into the PGPR, which is then monitored for viability and expression of the sequences contained in the vector. In addition, reference may be made to the relevant texts and literature, which describe expression vectors and their suitability to any particular PGPR.

The expression vectors of the invention must be introduced or transferred into the PGPR. Such methods for transferring the expression vectors into PGPRs are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the PGPR. Also, microinjection of the nucleic acid sequencers) provides the ability to transfect PGPR. Other means, such as lipid complexes, liposomes, and dendrimers, may also be employed. Those of ordinary skill in the art can transfect a PGPR with a desired sequence using these or other methods.

For identifying a transfected PGPR, a variety of methods are available. For example, a culture of potentially transfected PGPRs may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the PGPR is transformed with at least one expression vector. When only a single expression vector is used (without the addition of an intermediate), the vector will contain all of the nucleic acid sequences necessary.

Once the PGPR has been transformed with the expression vector, the PGPR is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

The present invention provides for a method for constructing genetically modified yeast PGPR of the present invention comprising: (a) introducing one or more nucleic acid comprising open reading frames (ORF) encoding the enzymes described herein wherein each is operatively linked to a promoter capable of transcribing each ORF to which it is operatively linked, and/or (b) optionally knocking out one or more of the enzymes described herein such that the modified PGPR does not express the one or more knocked out enzymes.

In some embodiments, the PGPRs are genetically modified in that heterologous nucleic acid have been introduced into the PGPRs, and as such the genetically modified PGPRs do not occur in nature. The suitable PGPR is one capable of expressing a nucleic acid construct encoding one or more enzymes described herein. The gene(s) encoding the enzyme(s) may be heterologous to the PGPR or the gene may be native to the PGPR but is operatively linked to a heterologous promoter and one or more control regions which result in a higher expression of the gene in the PGPR.

The genes or enzyme can be native or heterologous to the PGPR. Where the gene or enzyme is native to the PGPR, the PGPR is genetically modified to modulate expression of the genes or enzyme. This modification can involve the modification of the chromosomal gene encoding the gene or enzyme in the PGPR or a nucleic acid construct encoding the gene of the enzyme is introduced into the PGPR. One of the effects of the modification is the expression of the gene or enzyme is modulated in the PGPR, such as the increased expression of the gene or enzyme in the PGPR as compared to the expression of the enzyme in an unmodified PGPR.

In some embodiments, the PGPR is genetically modified in that heterologous nucleic acid have been introduced into the PGPRs, and as such the genetically modified PGPRs do not occur in nature. The suitable PGPR is one capable of expressing a nucleic acid construct encoding one or more enzymes described herein. The gene(s) encoding the enzyme(s) may be heterologous to the PGPR or the gene may be native to the PGPR but is operatively linked to a heterologous promoter and one or more control regions which result in a higher expression of the gene in the PGPR.

In some embodiments, each introduced enzyme can be native or heterologous to the PGPR. Where the enzyme is native to the PGPR, the PGPR is genetically modified to modulate expression of the enzyme. This modification can involve the modification of the chromosomal gene encoding the enzyme in the PGPR or a nucleic acid construct encoding the gene of the enzyme is introduced into the PGPR. One of the effects of the modification is the expression of the enzyme is modulated in the PGPR, such as the increased expression of the enzyme in the PGPR as compared to the expression of the enzyme in an unmodified PGPR.

In some embodiments, PGPR is a bacterium of the Rhizobium, Agrobacterium, Ralstonia, Allorhizobium, Azorhizobium, Bradyrhizobium, Ocrhobactrum, or Pseudomonas genus. In some embodiments, the bacterium is Azospirillum fluorescens or Azospirillum lipoferum. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, P. plecoglossicida, or P. simiae.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

### Synthetic Plant Growth Promoting Rhizobacteria Significantly Increased Plant Biomass Yield

The world needs sustainable agricultural solutions to meet ever-increasing demands for food and energy. Plant-growth-promoting rhizobacteria (PGPRs) may offer elegant solutions to this challenge, and using synthetic biology to engineer PGPRs can further amplify their efficacy. Here we demonstrate development of strategies for engineering to create PGPRs that modulate the effects of the plant hormones, auxin and ethylene. Auxin and ethylene are ideal targets, because their functions in plant growth and development are well documented. However, how plant-microbe interactions maintain the delicate balance of these hormones in plants is still unclear. To study this and develop engineering strategies, we implemented robust pathways for auxin biosynthesis and ethylene precursor degradation using Pseudomonas simiae WCS417 as a model, an elite PGPR that can robustly colonize diverse plant species and natively possesses bio-stimulant as well as bio-pesticide activities. Subsequent analyses suggested that plants largely control localization of bacterial auxin production via root exudates and that soil is essential to enhance this interaction. The engineered PGPR significantly enhanced shoot and root growth, and the growth enhancement is even better in an adverse condition. These results provide evidence that synthetic PGPRs can help reduce the negative effects of climate change and offer sustainable solutions to the world's most important problems related to food and energy production.

Here, therefore, we explore an alternative strategy to make agriculture more sustainable, microbiome engineering; that is, stacking important and complementary PGP traits to the existing elite PGPRs possessing ability to robustly colonize diverse crop species and to show various native PGP activities to further improve their eliteness. A similar approach is becoming more popular in medical applications to improve human health. For instance, a model probiotic strain, Escherichia coli Nissle, is often used as a chassis; this strain has been engineered to diagnose bleeding in the gastrointestinal tract and to treat metabolic diseases and cancers (Riglar et al., 2017). Similarly, in agriculture, companies such as Pivot Bio have been spearheading efforts to develop bacteria with enhanced ability to fix nitrogen (Rathi, 2018). Some government agencies have started programs to support efforts to safely use genetically modified microbes (GMMs) in the environment (Hanlon and Sewalt, 2020). In the near future synthetic biology may play critical roles in increasing agricultural production in the face of adverse effects from climate change.

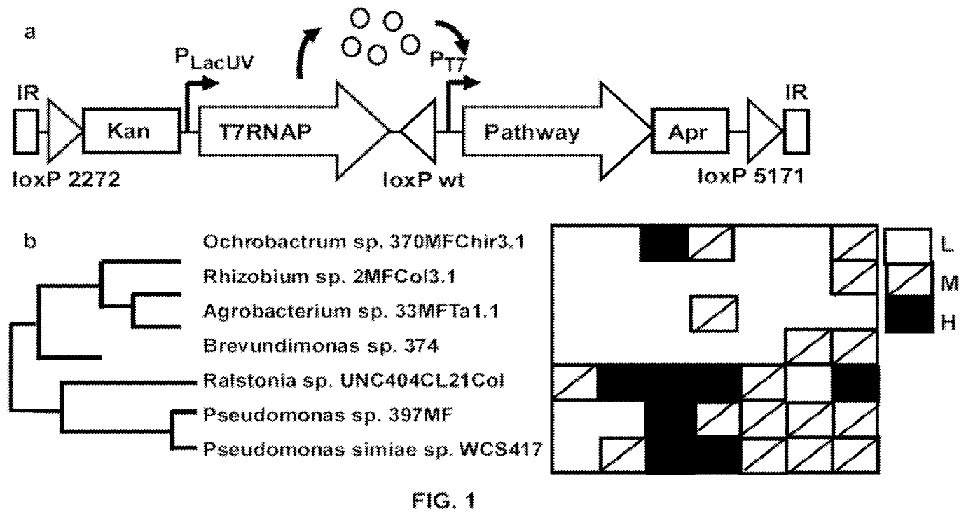

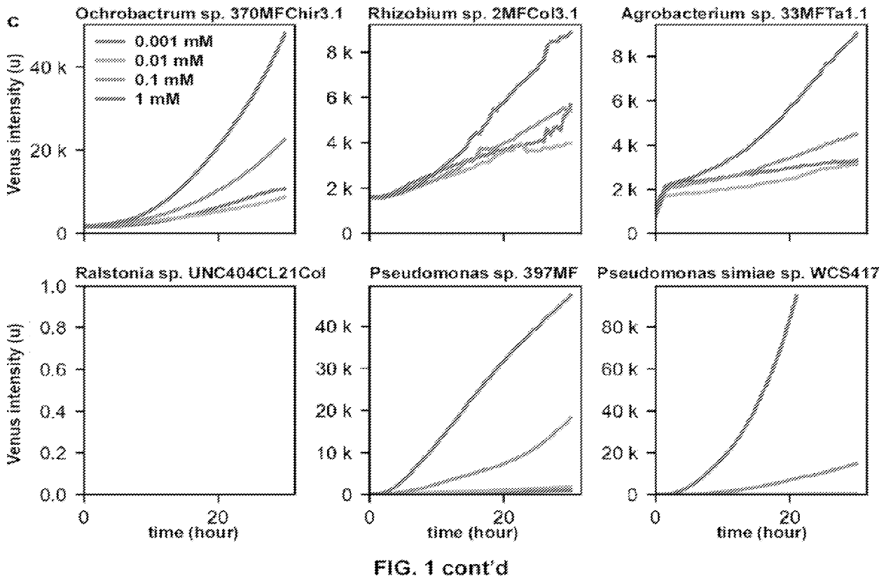

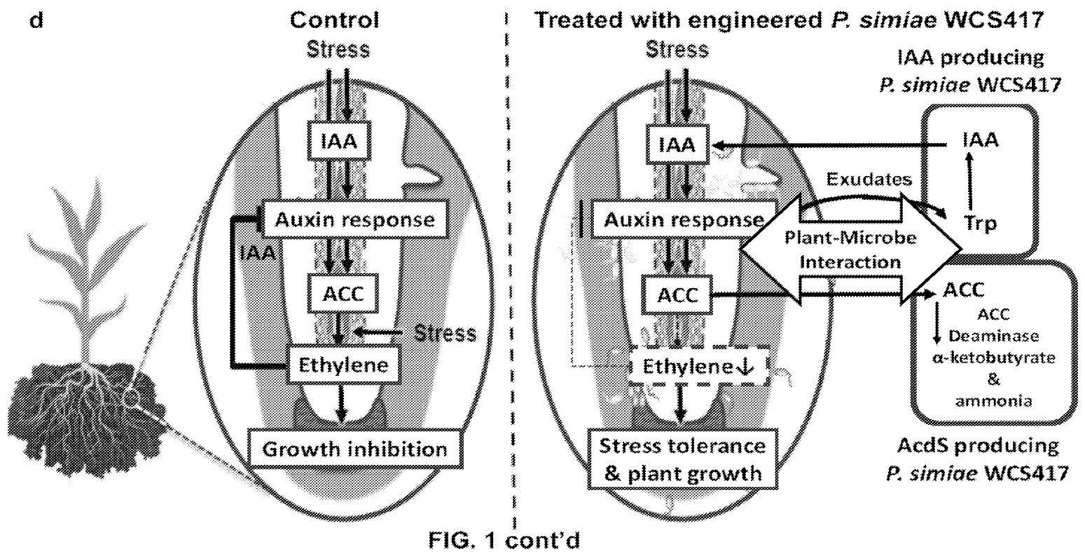

We selected PGP traits that manipulate the levels of auxins and ethylene in plants for this study. These phytohormones modulate plant growth and development and are reported to play important roles in plants' ability to tolerate both abiotic and biotic stresses (FIG. 1). Indole-3-acetic acid (IAA) is the main form of auxin in plants, “influencing almost anything” in plant growth and development. In addition, the IAA concentration gradient in roots is important for plant growth and development. IAA is enriched in the quiescent center (QC) of root meristem (Leyser, 2018), and both plants and bacteria maintain its gradient there in a delicate balance through control of IAA production, transportation, and degradation. When this balance is suboptimal, plant physiology is seriously disturbed. For example, an Arabidopsis mutant with IAA overproduction shows a super-root phenotype (Boerjan et al., 1995). Exogenous application of IAA initiates formation of lateral roots but inhibits elongation of primary roots (Takahashi, 2013; Barbez et al., 2017). IAA forms a feedback loop with ethylene: overproduction of IAA triggers ethylene production, which inhibits auxin responses and thereby primary root elongation as well as lateral root development (Dubois et al., 2018). Biotic and abiotic stresses also induce production of “stress ethylene,” which inhibits plant growth and development (FIG. 1). Stress ethylene can be reduced by breakdown of its intermediate, 1-aminocyclopropane-1-carboxylate (ACC), by ACC deaminase (acdS).

Based on these understandings, we formulated our design approach to develop PGPRs as to possess abilities to provide IAA to create an appropriate auxin gradient in the root systems and to efficiently degrade a precursor of stress ethylene. A number of PGPRs that can synthesize IAA are previously isolated from the rhizosphere, and growth improved in plants inoculated with them (Singh et al., 2015; Patten and R. Glick, 2002; Li et al., 2000). However, their abilities to produce IAA in the lab environments often do not correlate with their abilities to promote plant growth, suggesting that the PGP activity mediated by IAA is much more complex than what we generally screen for in the laboratory. Other factors such as PGPRs'(1) intrinsic and extrinsic regulations of IAA production, (2) other PGP traits stacked on IAA biosynthesis, as well as (3) colonization efficiencies and spatiotemporal behaviors may be important and need to. At the genetic level, plant-microbe interactions mediated by IAA is poorly understood. Among a few studies including deletion and overexpression of IAA biosynthesis pathways only resulted in moderate changes in plant growth (Patten and R. Glick, 2002) (Zúñiga et al., 2018). In contrast, the efficacy of acdS is relatively well more described. For example, Liu et al. surface-expressed acdS in three banana-endophytic strains. Compared with wild-type strains, the engineered strains reduced stress ethylene, significantly increased resistance to wilt, and increased plant yield (Liu et al., 2019). Therefore, better understanding of how the plant-microbe interactions mediated by IAA and the interplay between IAA and ethylene occur provides important guidance for us to establish a design principle, successfully conferring microbiome ability to promote plant growth mediated through IAA.

As a model chassis rhizobacterium strain to build up both IAA and acdS traits, we selected Pseudomonas simiae WCS417. WCS417 has several ideal characteristics to study aforementioned factors in IAA mediated PGP traits. First, our preliminary studies suggested that WCS417 neither showed IAA biosynthesis nor acdS activities, so it is easier to study intrinsic and extrinsic regulation of IAA biosynthesis and the interplay between IAA and acdS activities. Second, WCS417 is isolated from wheat roots for its elite plant-growth-promoting (PGP) activity nearly 30 years ago (Berendsen et al., 2015). This strain limits buildup of take-all disease and significantly increases wheat yield (Lamers et al., 1988). Further studies confirmed that WCS417 could induce systemic immunity in diverse plant species and confer resistance to a broad spectrum of diseases caused by both bacterial and fungal pathogens (Van Peer et al., 1991). WCS417 improves tolerance for abiotic stresses including drought and excessive salinity and can produce volatile organic compounds to induce auxin phenotypes (e.g., plants with greater growth of lateral roots and root hairs) as well as siderophores to acquire iron. Introduction of IAA and acdS traits can further improve its eliteness. Third, Pseudomonas species are among the best root colonizers and are generally enriched in the rhizosphere. Similar to other elite Pseudomonas species, WCS417 can robustly colonize roots of diverse crop species, and it forms biofilm throughout the root systems. Because of these PGP and colonization characteristics, WCS417 has long served as a model strain to study plant-microbe interactions and therefore can serve as an excellent model strain in our effort on microbiome engineering using synthetic biology. Lastly, new strains derived from this well-known microbe will be more amenable to regulatory approval for use in agricultural settings.

In this study, our goal is to engineer PGPRs that could promote plant growth under both normal and adverse conditions. For this engineering effort, we needed to carefully assess IAA biosynthesis and its regulation, interaction between WCS417 and plants, as well as the interplay between IAA and ethylene. The results of our studies allowed us to develop an engineering strategy to introduce IAA and acdS PGP traits in microbiomes to help increase agricultural production as we face climate change challenges.

### RESULTS

### Construction of a P. simiae WCS417 Strain That can Produce IAA

At least five IAA biosynthesis pathways have been proposed, all of which require tryptophan (Trp) as a precursor. Among these pathways, and well-characterized, are the indole-3-acetamide (IAM) pathway, the indole-3-pyruvic acid (IPA) pathway, and the indole-3-acetonitrile (IAN)/indole-3-acetaldoxime (IAOx) pathways (Patten and Glick, 1996; Duca et al., 2014a). These pathways are oxygen-dependent, as their key enzymes require molecular oxygen for their catalysis. In some water plants, oxygen is actively transported to the rhizosphere. However, it is not clear how extensively this transport mechanism is conserved in land plants, and its efficiency, robustness, and regulation are unknown. Since research is difficult if the engineered pathway depends on factors that are hard to manipulate, we selected the oxygen-independent IPA pathway as an engineering target.

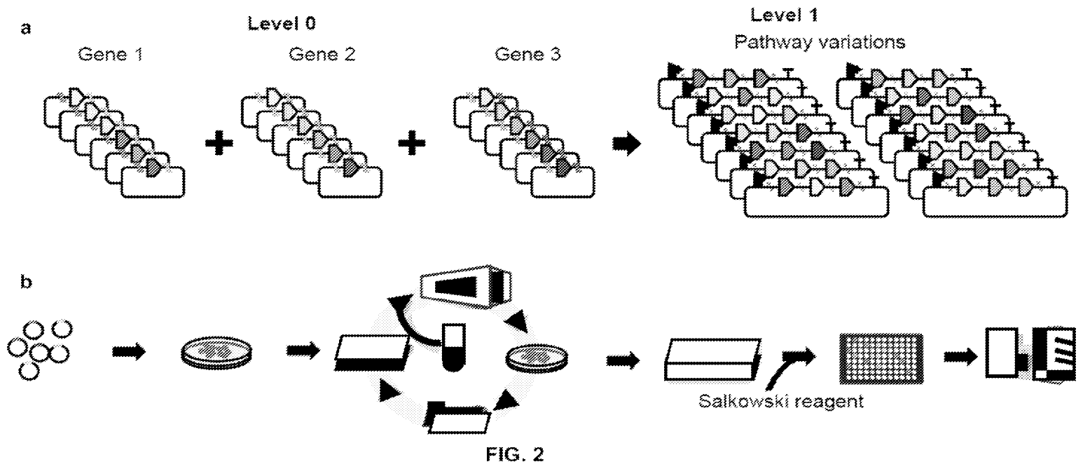

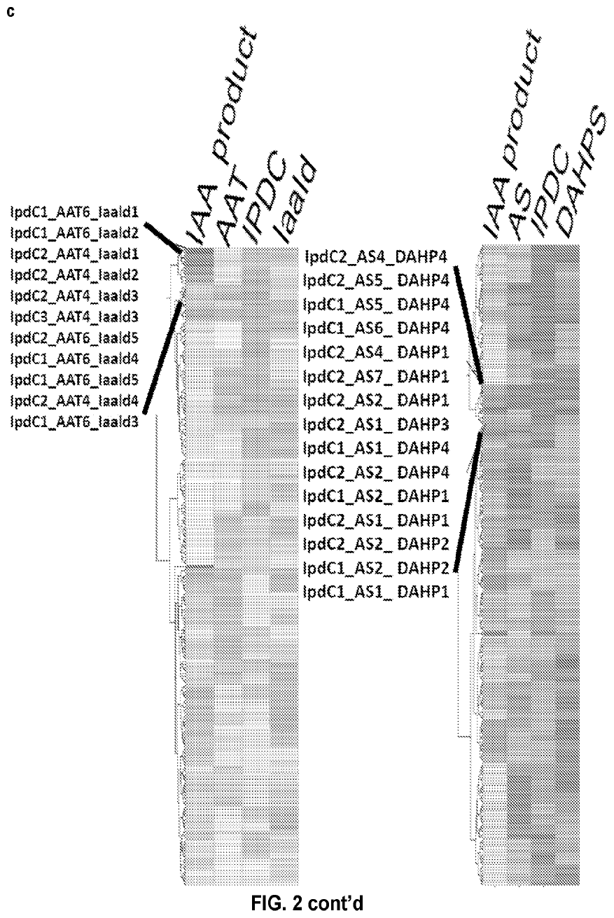

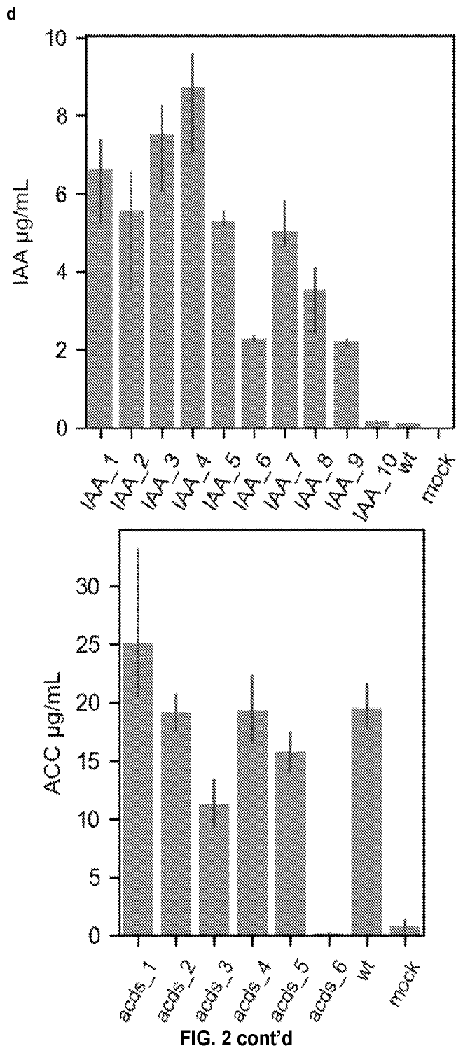

The IPA pathway is composed of three enzymes (Kasahara, 2016; Spaepen and Vanderleyden, 2016): tryptophan aminotransferase (TAT), indole-pyruvate decarboxylase (IPDC), and indole-3-acetaldehyde dehydrogenase (IAALD) (FIG. 2). Many bacteria isolated from the rhizosphere likely use this pathway to produce IAA. Although their ability to produce IAA is often limited under normal culture conditions, it is dramatically increased when they are cultured in rich media supplemented with the precursor Trp. Because Trp biosynthesis generally requires higher cellular resources than does other amino acid biosynthesis, IAA production is likely limited under normal conditions and is activated when more cellular resources are available. The genes involved in the IPA pathway are not clustered, unlike those of many other secondary metabolite biosynthesis pathways, including the IAM pathway. This may suggest that IAA production through the IPA pathway is regulated by multifaceted mechanisms. Alternatively, the IPDC pathway may be the only one that needs to be regulated, as TAT is part of Trp metabolism, and the IAALD reaction is catalyzed by promiscuous activity of ALD enzymes present in host microbial strains. With this in mind, we first sought enzymes that are involved in the IPA pathway, then refactored their genes and assembled them into two series of pathway variants.

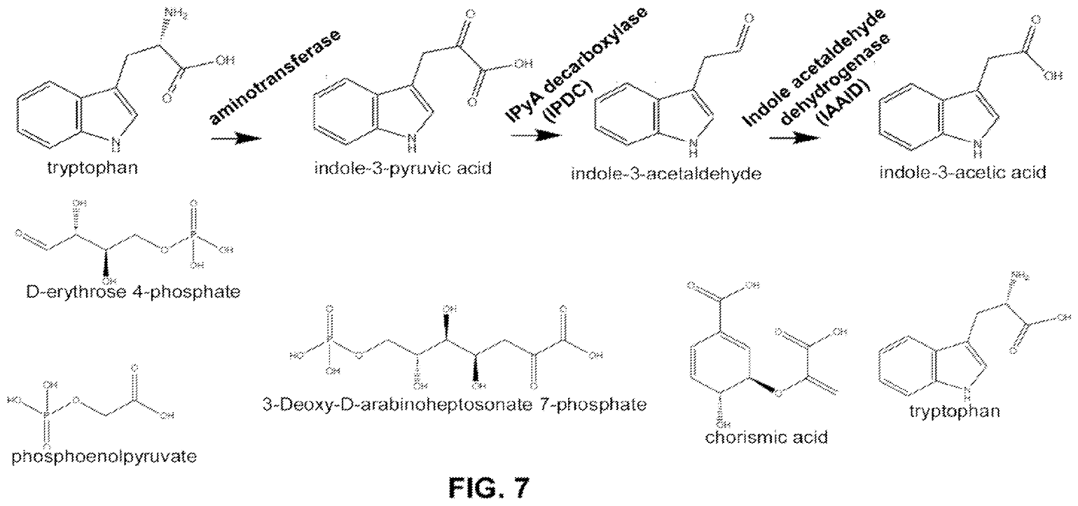

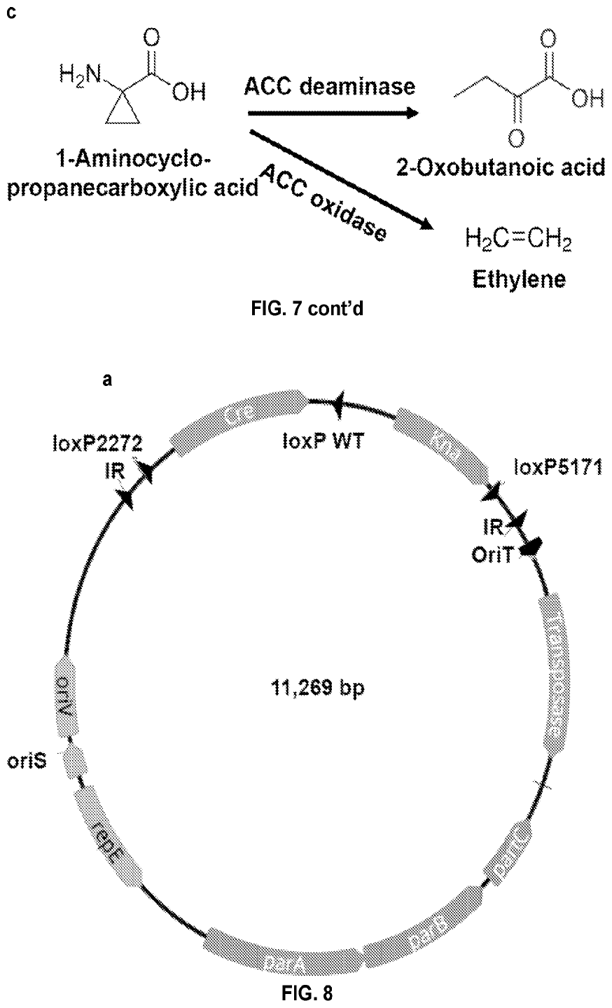

The first pathway (IPA1) comprises TAT, IPDC, and IAALD genes primarily targeting production of IAA from Trp. In contrast, the second pathway (IPA2) comprises 3-deoxy-D-arabino-heptulosonate 7-phosphate synthase (DAHPS), anthranilate synthase (AS), and IPDC genes. DAHPS and AS are rate-limiting enzymes for the shikimate pathway and Trp synthesis (FIG. 7). Overexpression of these enzymes may therefore enhance de novo synthesis of Trp, and thereby of IAA. While an IAA transporter might be necessary for IAA secretion, we are unable to find any research describing IAA transporters in bacteria and therefore decided not to include them in the design. To identify genes coding for high-performing enzymes in WCS417, we selected five to seven genes coding for previously identified enzymes to catalyze the reaction at each step of IPA1 and IPA2, using the enzyme database BRENDA (Jeske et al., 2019). Leveraging the U.S. Department of Energy Joint Genome Institute's (JGI's) DNA Synthesis Platform, we assembled combinatorial libraries for IPA1 and IPA2, each comprising over 200 pathway variants (FIG. 2). Subsequently, we integrated these pathway variants into the chromosome of WCS417 under control of the isopropyl-β-D-thiogalactoside (IPTG)-inducible lacUV5 promoter using CRAGE-Duet (FIG. 2). IAA is produced from each construct and measured in 96-well plate formats using the Salkowski method (Glickmann and Dessaux, 1995). Clustering analysis of the assay results indicated that the IPA1 pathway generally outperformed the IPA2 pathway in IAA production. We determined that IPDC is the key enzyme in both the IPA1 and the IPA2 pathways, and that high IAA producers are represented by IPDC1 and IPDC2 variants (FIG. 2).

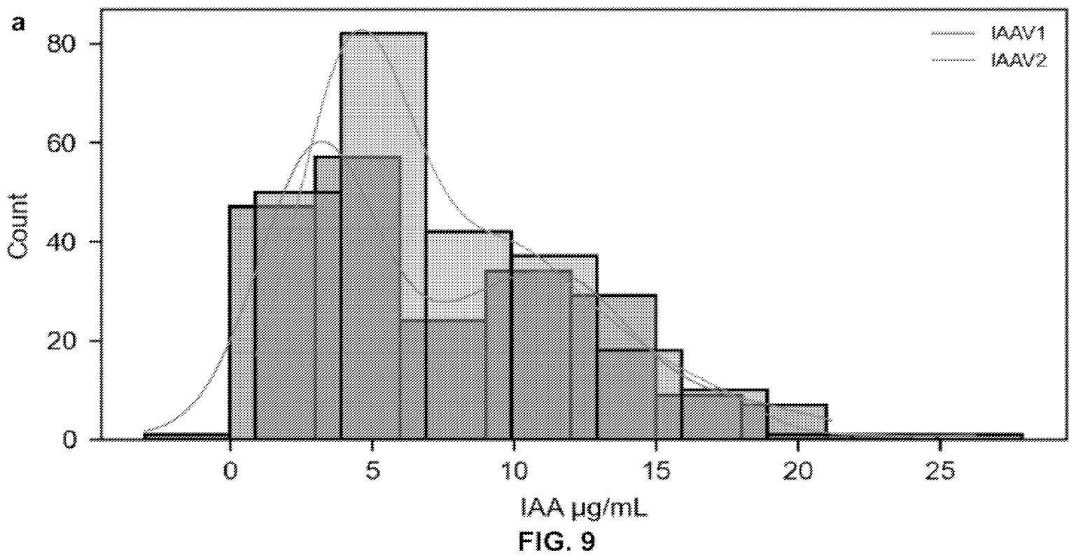

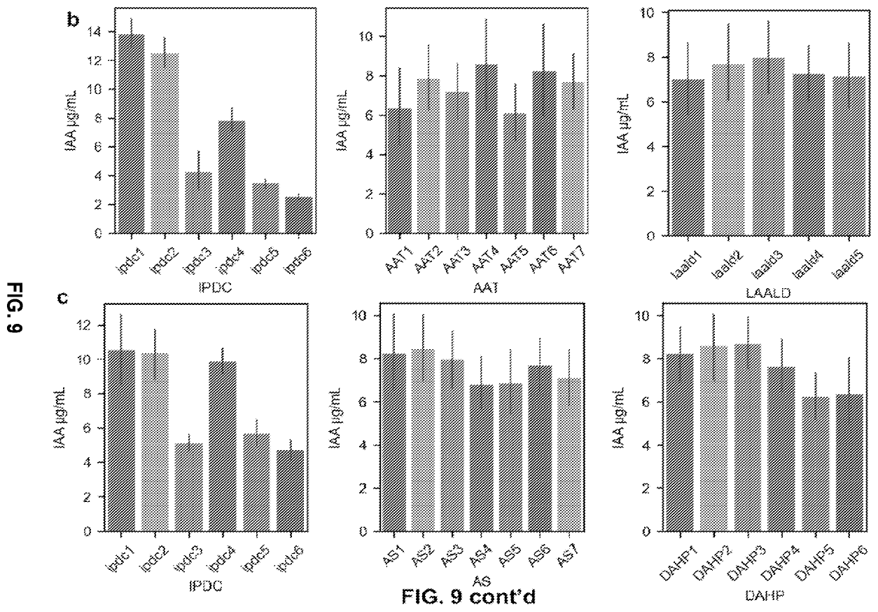

We repeated IAA production for the top six IAA producers under culture conditions more controlled than the screening conditions (FIG. 2, FIG. 9). Each culture is induced with different concentrations of IPTG ranging from 0 to 1 mM. The resulting IAA production is measured by high performance liquid chromatography-high resolution mass spectrometry (LC-MS). LC-MS confirmed production of IAA in the media from all six engineered constructs, but not from the WCS417 wild-type strain. The highest IAA production is 44 μg/mL, which is comparable to production among the highest IAA producers without Trp supplement previously reported.

### Construction of a P. simiae WCS417 Strain That can Degrade ACC

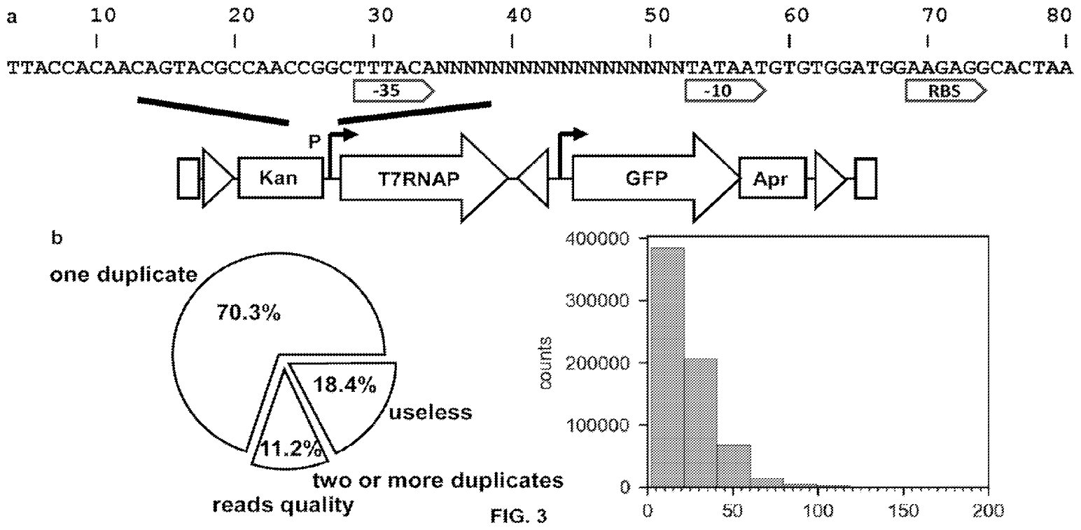

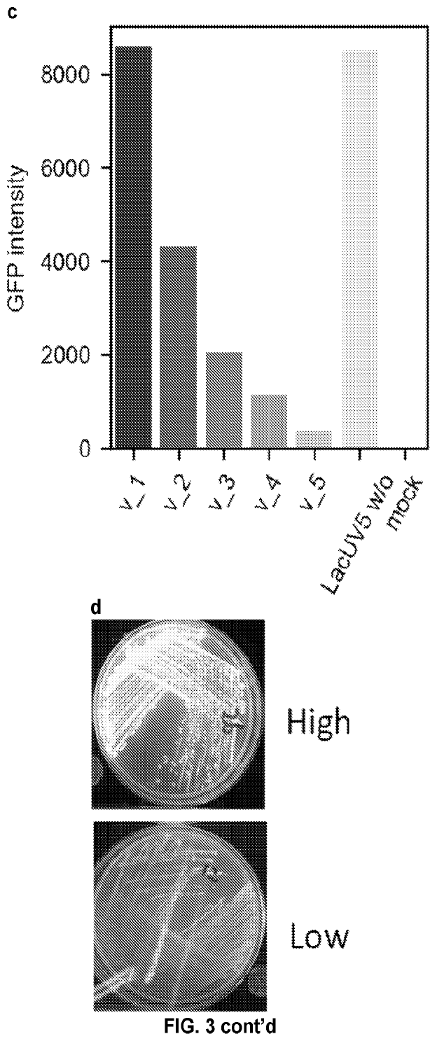

ACC deaminase (acdS) is an enzyme that can catalyze decomposition of ACC into a-ketobutylate and ammonia (FIG. 7). Plant-produced ACC is otherwise converted to ethylene, catalyzed by ACC oxidase. Excess production of stress ethylene because of biotic and abiotic stresses inhibits plant growth. AcdS is widely distributed among different species of rhizobacteria across multiple phyla. The current model is that ACC is excreted from plants to the rhizosphere, where it is taken up and degraded by acdS-producing bacteria. It is unclear whether a specific transporter is required for ACC uptake to the cytosol. Considering this model, we sought acdS that could give WCS417 the ability to efficiently degrade ACC. We selected six acdS candidates. We then synthesized the associated genes, assembled them, and integrated them into the chromosome of WCS417 using CRAGE-Duet under the control of the IPTG-inducible lacUV5 promoter. These strains are cultured in media containing 250 μM ACC. Only acdS6 (from Phytophthora sojae) could efficiently degrade ACC (FIG. 3).

We subsequently tested the ACC degradation activity of WCS417 expressing acdS6. We repeated the experiment and did a time-course analysis under culture conditions more controlled than are the screening conditions (FIG. 3). Each culture is induced with different concentrations of IPTG, ranging from 0 to 1 mM. While the WCS417 wild-type strain did not have acdS activity, the engineered strain expressing acdS6 is able to quickly decompose ACC. Within 24 hours after the culture is initiated, 3.2 mM of ACC is completely digested. The ACCD activity of this engineered strain is comparable to that of natural isolates screened and isolated for ability to efficiently degrade ACC. Interestingly, IPTG concentration did not greatly affect ACC degradation. This may suggest that aspects other than acdS activity, such as ACC transportation, might be the rate-limiting step for ACC degradation in this engineered WCS417 strain.

### Identification of Promoter Variants With Different Activity for Production of IAA and acdS

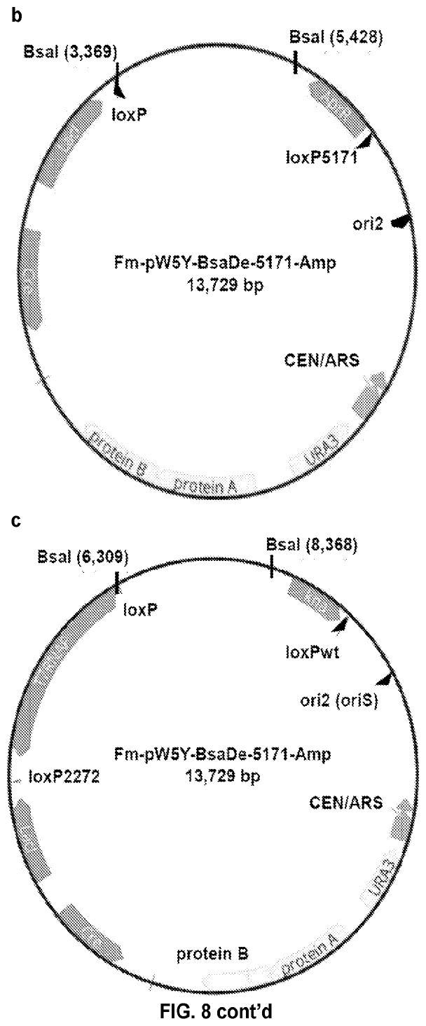

Plant roots are highly sensitive to the concentration of the phytohormones IAA and ethylene (Okumura et al., 2013). We are interested in studying dose-dependent responses of plants to different IAA and acdS activities and their combinations. To alter IAA and acdS activity, we used a set of promoters with different expression strength. Because WCS417 is not a model strain for metabolic engineering, well-characterized genetic parts are not readily available. Therefore, we explored the variants of an E. coli lacUV5 promoter (Supplementary FIG. 8). The region between −35 and −10 of this promoter is essential for its activity (Bai et al., 2015). To identify constitutive promoters of diverse strengths, we modified the lacUV5 promoter by removing its LacI operator motif and replacing the region between 35 and −10 with random nucleotides. To screen the strength of these promoters, we built a construct using the CRAGE-Duet system. We integrated green fluorescent protein (GFP) under the control of the T7 promoter and terminator into the first integration site, and T7 RNA polymerase (T7RNAP) under the control of a library of lacUV5 promoter variants into the second integration site. In this way, we could modularize expression control of the PGP pathways, so the identified promoter constructs could be readily used to express both the IAA biosynthesis pathway and acdS.

High throughput sequencing analysis identified 4,970,500 unique promoter sequences in the library, 70% of them occurring as singletons. Sequences that occurred twice or more accounted for 11%; the average read count of these is 28 (FIG. 11). The remaining sequences did not match any of the designed sequences. This analysis suggests that the library has tremendous sequence diversity. Fluorescence-activated cell sorting (FACS) indicated that many of the sequence variants lost promoter activity relative to the wild-type lacUV5 promoter activity. However, the promoter variants in the library gave broad GFP activity, ranging from 200 to 10,000 units (FIG. 11). Interestingly, bases toward both ends of the promoter variants with high expression activity tended to be more conserved than those with medium and low expression (Supplementary FIG. 11). In particular, we found the base at the 16th position is strictly conserved as “T” for promoters with strong activity. We subsequently selected two promoter sequences with high and low expression strength, and integrated them into the second location of the CRAGE-Duet system to drive expression of the IAA biosynthesis pathway and acdS.

We tested both IAA biosynthesis and acdS activities (FIGS. 2 and 3). For the IAA biosynthesis, the IAA producer driven by strong (s.IAA), medium (m.IAA), and weak (w.IAA) promoters produced 20, 15, and 10 μg/mL (FIG. 2), respectively. We have decided to use the s.IAA and w.IAA WCS417 strains for subsequent studies. Additionally, it is also our interest to understand If this IAA pathway allows IAA production from other substrates than Trp as our minimum media is supplemented with 5 g/L of yeast extract, which may contain up to 200 μM. While the engineered IAA producer can produce up to 250 μM IAA, but this only provides indirect evidence. Therefore, we fed 13C labeled glucose-13C6 to the media and tested their ability to produce IAA from glucose. The LC-MS analysis suggested that the engineered WCS417 strain can indeed produce IAA directly from the fed glucose as well as the fed glucose and other ingredient of the media, and they are estimated to correspond 38% and 25% of the total IAA produced, respectively. For the acdS expression, acdS driven by strong (s.acdS), medium (m.acdS), and weak (w.acdS) are built. These strains are able to efficiently degrade ACC and their activity is comparable to each other and to those induced by the wild type lacUVS promoter with different IPTG concentrations.

### The Effects of the Engineered WCS417 IAA Producers are Regulated by Plants

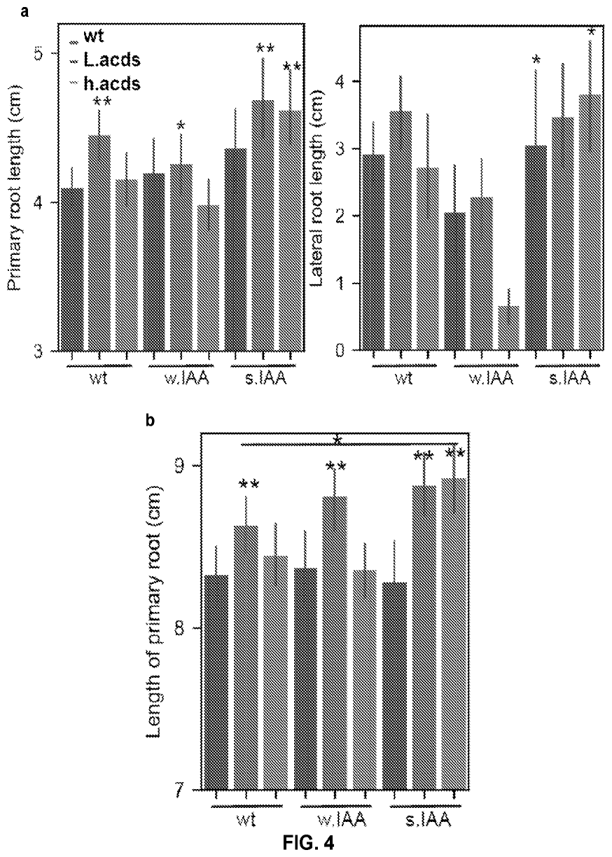

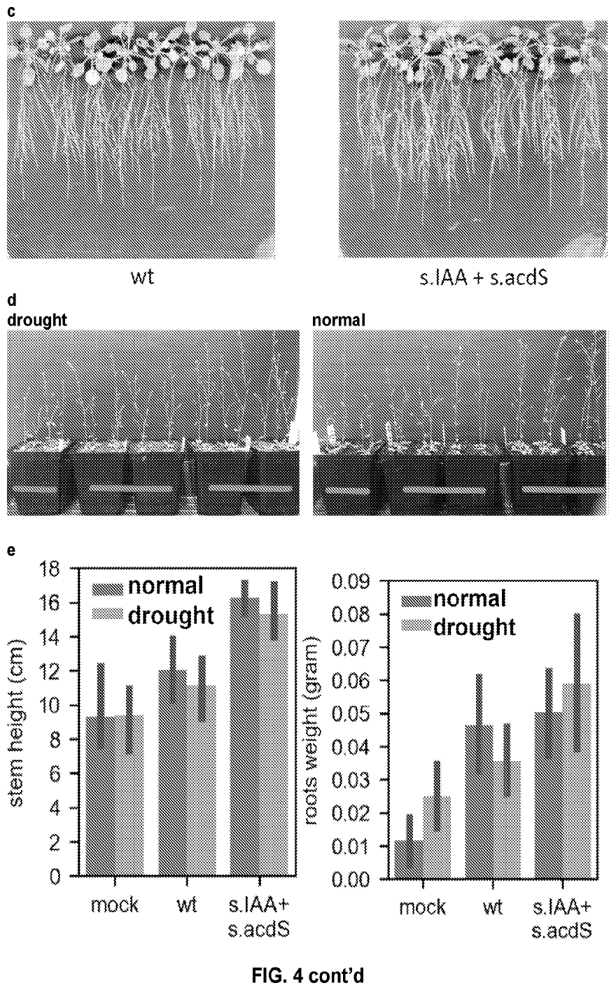

To investigate the dose-dependent response of plants relative to different IAA production and acds activity, we combinatorically treated Arabidopsis plants with engineered strains with two different promoter strengths for each trait and investigated the strains' effects on plant-growth promotion. We characterized shoot and root morphologies and weights (FIG. 4). As expected, plants treated with the WCS417 strain showed the auxin phenotype which is induced by VOCs; while primary root growth is inhibited, lateral root growth is significantly improved. Both shoot and root weights are increased. However, engineered PGP traits did not affect plant-growth phenotypes much. Expression of w.acdS constantly increased both primary and lateral root growth as well as root weight. However, growth promotion is limited to 10% or less (FIG. 4). Inoculation with IAA-producing strains did not promote the growth of Arabidopsis except when the s.IAA strain is paired with the s.acdS strain. Again, growth promotion is limited to 10% or less (FIG. 4).

To further study interactions between plants and the engineered WCS417 strains, we inoculated Arabidopsis mutants possessing an IAA biosensor (DR5rev::3xVENUS) with the engineered WCS417 IAA producers. This Arabidopsis mutant line has the DR5rev::3xVENUS reporter system which is controlled by auxin responsive promoter, and this reporter line allows us to study the effect of the engineered strains' IAA production in situ. Arabidopsis roots are extremely sensitive to externally added IAA; even at the 0.01-0.1 μM level, IAA significantly changes root phenotypes. It has been reported that IAA reporter lines of Arabidopsis shows auxin dependent DR5 expression in the middle of apical meristem toward the quiescent center (QC). When Arabidopsis is treated with 1 μM or more of IAA, it starts to show severe inhibition of root growth and development. Under this condition, the roots of the IAA biosensor line of Arabidopsis showed the lost IAA gradient and are saturated with IAA. Because our strains can produce 20 μg/mL (115 μM) IAA in culture media, in theory we would expect to see IAA saturation throughout the root system. Interestingly, the DR5rev::3xVENUS line inoculated with the IAA producer did not show saturation of signal. Instead, the signal is localized from QC to initial cells and epidermis.

This result suggests that plants likely regulate bacterial IAA production in a way that maintains the IAA gradient in their root systems. This phenomenon may be explained well by our previous studies monitoring the Brachypodium root colonized with the WCS417 strain labeled with bacterial luciferase. Bacterial luciferase comprises an operon of five genes and requires ATP, NADPH, and FMNH₂ for luminescence. Therefore, it is an ideal marker for the metabolic activity of the WCS417 strain colonizing different locations in the root system. Interestingly, we saw a bright luminescence signal around each root tip, and a faint signal around the more mature root systems. We repeated this experiment with Arabidopsis and saw a similar pattern for the metabolic activity of the WCS417 strain (FIG. 4). This pattern is likely created by a difference in the amount of root exudates provided between the root tips and the more mature root areas and is clearly ideal for plant roots to create the IAA gradient with the help of bacteria.

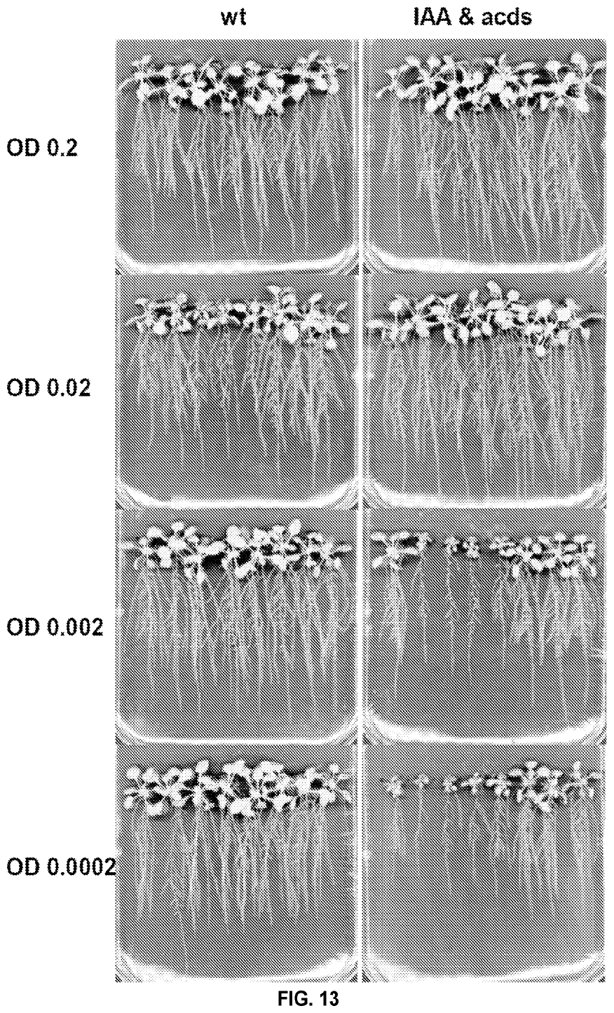

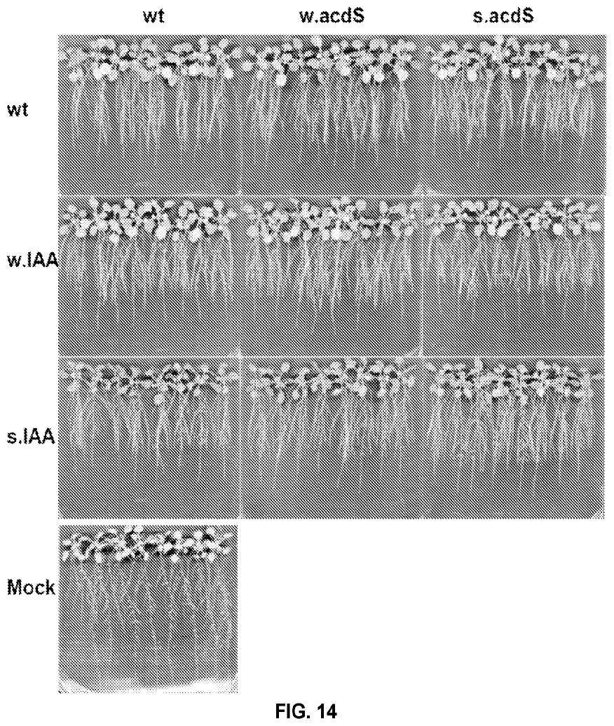

To further test this hypothesis, we supplied plant growth media with Trp (FIG. 4, FIGS. 13 and 14) and repeated the combinatorial assay. If the root-colonizing WCS417 strains are metabolically active throughout the root system, plant growth would likely be significantly impaired. In contrast, root growth might improve if the WCS417 strain are metabolically active only around the root tips. We did not see much impact on root growth at 0.1 μM of Trp. However, at 10 μM, Trp inhibited the root growth of plants colonized with the wild-type WCS417 strain. Both s.IAA and s.acdS ameliorated this stress and significantly improved root growth. The s.IAA and s.acdS combination further improved the plants' root growth phenotype. Additionally, we inoculated the engineered strains to the Trp-overproducing Arabidopsis mutant line. Root growth and development of this Arabidopsis mutant line are not also impaired (FIG. 4).

These results suggest that the IAA biosynthesis pathway under the strong constitutive promoter in WCS417 remains active, and plants are likely controlling the WCS417 strain's ability to produce IAA via their root exudates. Although multiple genetic circuits for regulating bacterial IAA biosynthesis are previously identified, our result indicated that these circuits exist for bacteria to simply save resources and energy while they survive in soil rather than to control and maintain the IAA concentration gradient in root systems.

### Interactions Among Plants, Microbes, and Soil are Important for IAA Activity

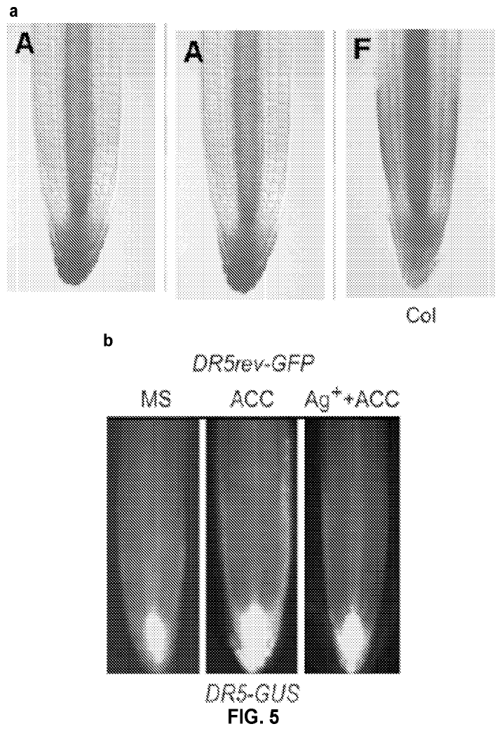

As another way to study the effect of IAA on root growth and development, we inoculated various IAA-deficient Arabidopsis mutant lines with the IAA-producing WCS417 strain and grew the plants on agar media. The engineered WCS417 strain did not improve the growth of Arabidopsis IAA mutants grown on agar media. We subsequently transferred one of the IAA mutant lines to soil. This Arabidopsis mutant line contains triple mutations (wei8-1 tar 1-1 tar 2-1), is insensitive to ethylene, and has a severe deficiency in auxin biosynthesis (Stepanova et al., 2008). Interestingly, this triple mutant grew significantly better when treated with the engineered IAA-producing WCS417 strain than when treated with the wild-type WCS417 strain (FIG. 5). This result suggests that interactions among plants, microbes, and soil are important for IAA activity; soil enhances IAA-mediated interactions between plants and microbes.

Considering this observation, we investigated whether our engineered strains could improve growth and development if plants are grown in soil. We first grew Arabidopsis for 2 weeks on agar media, colonizing the plants with either the wild-type WCS417 strain or the engineered s.IAA and/or s.acdS WCS417 strains. We measured plant growth phenotypes at 3 weeks after these plants are transferred to pots. The soil in the pots is non-sterile. In this way, we could also evaluate the robustness of the engineered strains for root colonization. Growth and development are dramatically improved for plants treated with either the wild-type or the engineered WCS417 strain. For Arabidopsis treated with the wild-type WCS417 strain, stem length increased by an average of 20%, and root weight increased by as much as 500% compared with the non-inoculated control. For Arabidopsis grown with s.IAA and s.acdS WCS417 strains, stem length is further elongated by 35%. Root weight remained the same quantity compared with growth of plants inoculated with wild-type WCS417.

We further tested the same experiment under drought conditions (reduced watering frequency). In response to drought stress, root/shoot ratio are increased This phenomenon is similar to responses reported elsewhere. Root and shoot biomass are increased for plants inoculated with the wild-type WCS417 strain. Growth is further enhanced when we inoculated with s.IAA and s.acdS WCS417 strains; root and shoot biomass are increased.

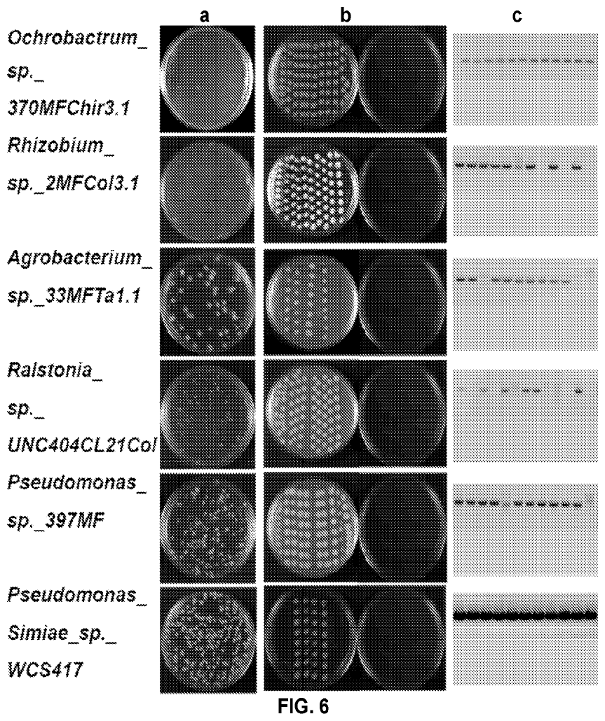

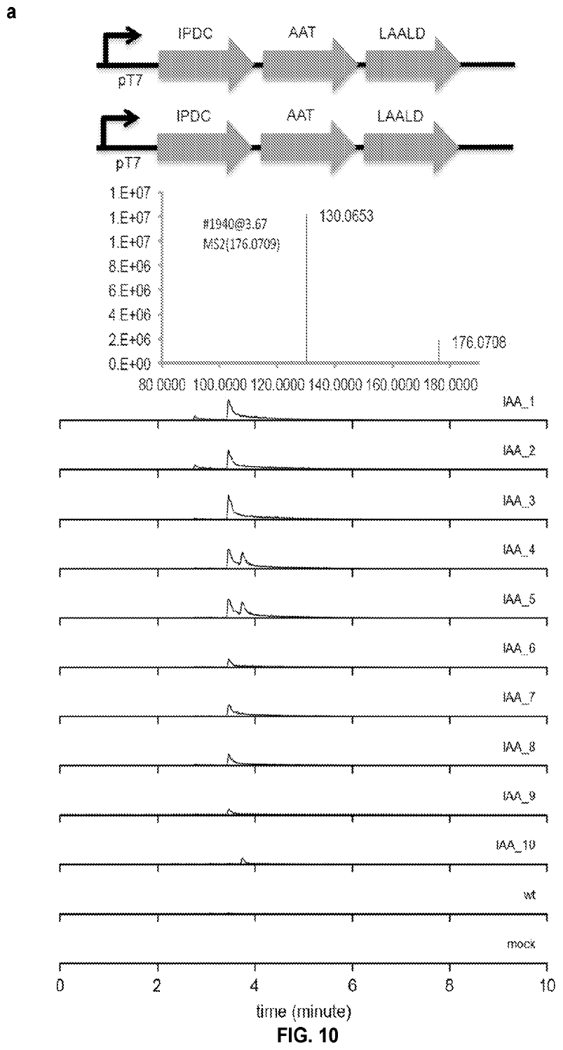

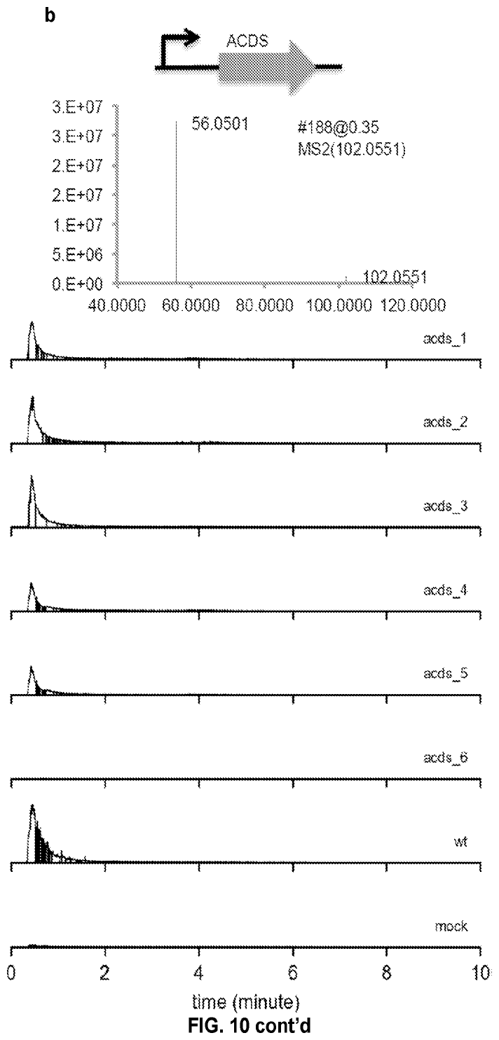

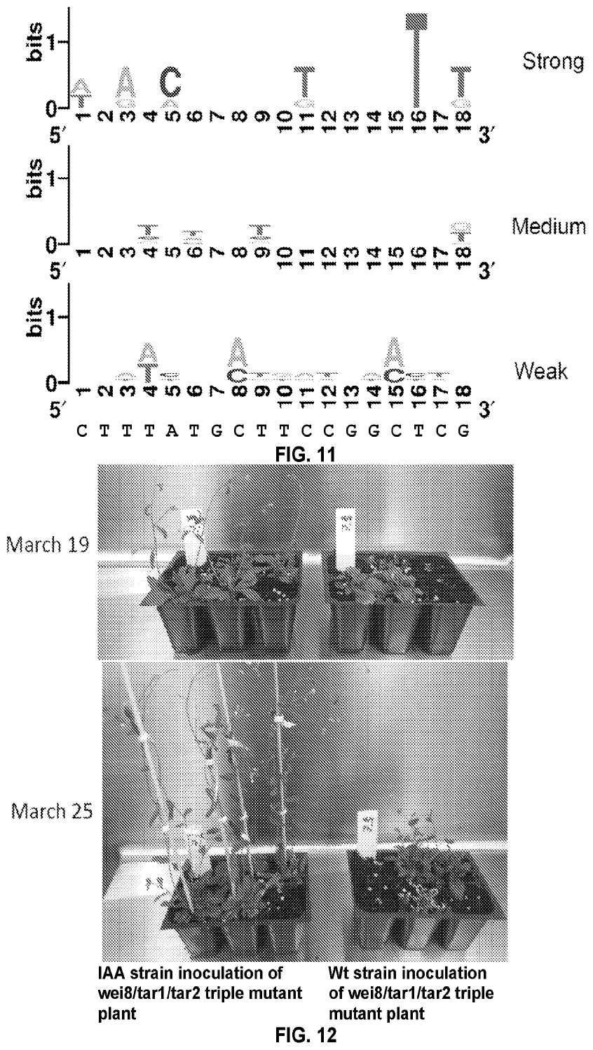

Results are further depicted in FIGS. 6-14.

### DISCUSSION

The world is facing ever-increasing demands for sustainable agriculture for energy and food crop production. At the same time, scientists predict that ongoing climate change will continue to adversely affect agricultural production. While PGPRs may have tremendous potential to help increase crop yields, their effectiveness varies, limiting their usefulness.

An ideal way to tackle the dual global challenges of increased demand (resulting from expanding population coupled with rising standard of living) and decreased yield (resulting from climate change) may be engineering of elite PGPRs so that they can robustly colonize the roots of diverse crop species. Synthetic biology may be an efficient way to modify these elite PGPRs by stacking useful PGP traits so these PGPRs can thrive in spite of the native biome's exclusion mechanisms. Therefore, we explored further extending the utility of an elite PGPR, P. simiae WCS417, which possesses several interesting PGP traits natively. We also reasoned that regulatory approval for field application would likely be faster if we engineered one or several well-characterized elite PGPRs than if we engineered members of a broader rhizobacteria community.

To enable higher biomass yield, particularly under adverse conditions (e.g., drought, heat, and high salinity), we introduced abilities in the WCS417 strain to efficiently produce IAA and acdS to manipulate the plant hormones IAA and ethylene. IAA and ethylene influence diverse aspects of plant growth and development. Expression of acdS in bacteria is mainly regulated by local ACC concentration. This makes sense because the primary role of acdS is to degrade excess ACC and mitigate growth inhibition caused by stress ethylene. However, regulation of IAA biosynthesis in bacteria is more complex. In some cases, Trp, Phe, and/or Tyr function as inducers. IAA can also serve as a positive feedback regulator. Specialized metabolites such as 2,4-diacetylphloroglucinol and phenyl acetic acid are also known to regulate IAA biosynthesis. In the case of IAA, its concentration gradient in roots is important, so plants are very sensitive to externally supplied IAA even at low concentrations.

Therefore, we anticipated that complex engineering design would be required for the WCS417 strain to supply the right amount of IAA at the right root locations. Our strategy is originally to build a WCS417 strain that could produce high levels of IAA, then tune down the IAA production using one of the genetic circuits. We thought adapting the IAA positive feedback loop might be the most logical way to help maintain the concentration gradient in roots. However, our results suggest that plants have their own ability to control bacterial IAA production; that is, the strength of IAA expression in bacteria likely depends less on the bacteria themselves than on root exudates from the plant that control bacterial metabolic activity and thereby IAA production. The genetic circuits present in the bacteria may exist simply to allow the bacteria to conserve energy so they can survive in resource-scarce environments. It would be interesting to further study the importance of having a genetic circuit to control expression of the IAA biosynthesis pathway for competitiveness of the WCS417 strain in the presence of other microbial species.

In addition to our discovery of plants' ability to control bacterial IAA synthesis, another important discovery of our work is that plant growth promotion by bacterial IAA is significantly enhanced in the presence of soil. Interestingly, this effect is amplified even further under drought conditions. This change may be attributed to limited diffusion of plant exudates or IAA in soil and/or simply to physiological changes in the plants. The molecular mechanisms are yet to be elucidated in detail. In our experiments, we first gave sufficient time for WCS417 strains to colonize the roots on agar plates. However, growth promotion is seen after the plants are transferred in pots containing non-sterile soil, suggesting that WCS417 is not eliminated from the native community.

Our next goal is to further investigate the effectiveness of the engineered strains for production of crops that are more relevant to energy and food security and to perform these experiments in more agriculturally relevant environments. Additionally, we note that engineered microbes can facilitate development of advanced root systems, which can produce larger soil organic matter (SOM). SOM improves soil structure, water retention, and fertilizer use efficiency, while preventing topsoil erosion. Advanced root systems may also increase CO₂ storage in soil, according to the US Department of Energy Advanced Research Projects Agency-Energy (ARPA-E). With so many potential roles for PGPRs, our results suggest that microbiome engineering using synthetic biology has tremendous potential to help advance sustainable and robust agriculture.

### METHODS

### Microbial Strains and Plant Lines

The Pseudomonas simiae sp. WCS417 strain is obtained from Prof. Jeffrey Dangl's group (University of North Carolina at Chapel Hill). Arabidopsis ecotype Col-0 (CS7000) and transgene plants including DR5rev::3xVENUS (CS799364), EBS::GUS (CS69046), and triple-mutant wei8-1 tar1-1 tar2-1(CS16419) are obtained from the Arabidopsis Biological Resource Center (ABRC).

### Vector Construction

To clone genes and pathways, a vector, pW5_5171_Bsa, is built. The BsaI site is first removed from vector pW5Y_Apr (Apr stands for apramycin resistant gene), and the vector is digested by the NotI enzyme for cloning a fragment of the Amp gene of vector pBAMI (Addgene, Cambridge, MA) to make vector pW5_5171_Bsa. To clone the specific promoter that would drive the T7RNAP vector, pW5_2272_T7_Bsa is built. This plasmid is constructed by joining the amplified fragments of R6K_2272_T7 (primers R6k-2272-T7-F/R) with the amplified fragments of BsaI-deleted pW5Y_Apr (primers pW5-BB-F/R). All enzymes and Gibson assembly kits are from NEB (New England Biolabs, Ipswich, MA).

### Combinatorial Assembly of IAA Biosynthesis Pathways

Candidate genes encoding Trp aminotransferase (TAT), indole pyruvate decarboxylase (IPDC), indole-3-acetaldehyde dehydrogenase (IAALD), 1-aminocyclopropane-1-carboxylate (ACC) deaminase, 3-deoxy-D-arabinoheptulosonate 7-phosphate synthase (DAHPS) and anthranilate synthetase (AS) are selected using BRENDA (webpage for: brenda-enzymes.org). Five to seven genes are selected for each enzyme for DNA synthesis. Synthetic genes are designed using BOOST (webpage for: boost.jgi.doe.gov); they are fused with a T7 promoter and RBS, and flanked by BsaI sites. These synthetic genes are obtained from Twist Biosciences.

The synthetic genes are amplified and cloned in the BsaI digestion site. Combinatorial assembly of the IAA synthesis pathways is performed using the Golden Gate assembly strategy (Marillonnet and Grützner, 2020). First, parts are joined into the intact gene by cloning relevant parts into vector JGI vector using a Gibson assembly kit (New England Biolabs, Ipswich, MA). Second, entire genes are released from vectors by Bsal digestion, and vector pW5_5171_Bsa is also digested with Bsal. Sets of digested genes and vectors are mixed and matched using an Echo acoustic liquid handling machine (Labcyte Inc., San Jose, CA) for T4 DNA ligase-assisted ligation. Ligation products are transformed into TOP10 chemical competent cells, and the transformed cells are plated on 48-well bioassay LB agar plates supplemented with 100 μg/mL of Apr (Molecular Devices, San Jose, CA). Positive clones are picked for plasmid isolation. Isolated plasmids are transformed into chemical-competent conjugation E. coli donor strain WM3064. All constructs are sequenced using the JGI PacBio sequencing platform for confirmation.

### Conjugation

The CRAGE-Duet system is used to integrate genes and pathways into P. simiae WCS417 (Wang et al., 2020). Briefly, in the first round of conjugation, WM3064 strains, each carrying different IAA biosynthesis pathways or acdS genes, are loaded onto 96 deep-well plates with 100 μg/mL Apr LB for incubation. A CRAGE-Duet-compatible conjugation recipient P. simiae WCS417 strain is prepared from the overnight culture grown in LB media supplemented with 100 μg/mL Kan at 28° C. The ex-conjugants are plated on 48-well bioassay square plates containing LB agar plates supplemented with 150 μg/mL Apr for selection. Three positive colonies are chosen from each conjugation reaction, and genomic integration of genes and pathways are confirmed by simple counterselection (positive to Apr selection/negative to Kan selection). The second round of conjugation is performed using conjugal E. coli donor strains carrying pW5_2272_T7_Bsa to each strain built above. The ex-conjugants are plated on 48-well bioassay square plates containing LB agar plates supplemented with 150 μg/mL Kan for selection. All conjugation procedures are assisted by automation equipment including Microlab VANTAGE Liquid Handling System (Hamilton, Reno, NV) and QPix 400 Series Microbial Colony Pickers (Molecular Devices, San Jose, CA).

### Salkowski Assay

A Salkowski assay is a colorimetric method to measure IAA concentration in microbial cultures (Glickmann and Dessaux, 1995). The Salkowski solution is prepared by mixing 2 mL of 0.5 M FeCl₃, 49 mL of water, and 49 mL of 70% perchloric acid in 100 mL solution. The WCS417 strains carrying different IAA synthetic pathway variants are first cultured in 100 μL of LB containing 150 μg/mL of Kan in Nunc™ 96-well microplates using an incubation shaker overnight. From each well, 5 μL of overnight culture is transferred into 0.8 mL modified M9 medium (Wang et al., 2019) containing 150 μg/mL Kan and 0.1 mM IPTG in a 96-well deep well plate and is cultured for 3.5 days at 28° C. These cultures are centrifuged for 15 min at top speed in a bucket rotor in an Eppendorf 5810R (Eppendorf, Hamburg, Germany). From each sample, 50 μL of supernatant is transferred to a 96-well black polystyrene microplate (Corning Inc, Corning, NY). To each well, 150 μL of freshly prepared Salkowski reagent is added. The black microplates are slightly vortexed and incubated at room temperature for 30 min. The absorbance at 530 nm is read using a Synergy HTX multi-mode microplate reader (BioTek, Winooski, VT). A standard curve is created using a serial dilution of IAA chemical compound (Sigma Aldrich, St. Louis, MO).

### LC-MS Assay

Following the same procedure for incubation as for the Salkowski assay, 0.5 mL of supernatant from every sample is lyophilized and added to 500 μL of 100% LC-MS-grade MeOH. Sample plates are first sealed with the plate sealer and then sonicated for 10 min. After centrifugation at 5,000 rpm for 5 min, supernatants are transferred into new 96-well deep well plates, while the pellets are left behind in the first plates. New plates are put in an SPD131DDA SpeedVac™ concentrator (Thermo Fisher Scientific, Waltham, MA) for evaporation. Subsequently, 150 μL of LC-MS-grade MeOH containing 1 μg/mL ABMBA is added to each well. Plates are sonicated for 10 min and then are centrifuged for 5 min at 5,000 rpm. Supernatants are transferred to Millipore filter plates (MilliporeSigma, Burlington, MA) over a new 96-well deep well plate for 2.5 min and centrifuged at 2,500 rpm. Sample plates are sealed in a plate sealer and are ready for running in a Q Exactive LC-MS (Thermo Fisher Scientific, Waltham, MA). Each treatment had three biological replicates. Generated MS data are analyzed by an MZmine2 (Pluskal et al., 2010) in comparison with IAA and ACC standards (Sigma Aldrich, St. Louis, MO). Construction of lacUV mutant library

To create a constitutive promoter for the lacUV5 mutant library, primers to delete the LacI operator motif and to degenerate a region between −35 and −10 are synthesized at IDT (Integrated DNA Technologies, Inc., Coralville, IA) (FIG. 3). 1 μL of 10 mM of this degenerate primer is amplified for 10 cycles by primer set. Then the purified product is cloned into Bsal-digested vector pW5_2272_Bsa using the Gibson assembly kit and 1 μL of the Ligation product is transformed into competent Top10 (Invitrogen, Carlsbad, CA) cells by electroporation at a voltage of 1.8 kV using a Biorad Pulser Xcell (Bio-Rad Laboratories, Hercules, CA). Transformants are plated on 48-well square bioassay plates supplemented with 100 μg/mL Apr and incubated overnight at 37° C. Colonies are collected by adding 10 mL sterile water per plate and scraping. 5 mL of cell collection is used for plasmid isolation with plasmid mid prep kit (Zymo Research, Irvine, CA). 5 μL of 200 ng/μL of isolated plasmid is transformed into 100 μL of electro-competent WM3064. Transformants are plated on 48-well square plates supplemented with 100 μg/mL Apr. Colonies are collected by scraping the plates. Conjugation is subsequently set up by mixing 100 μL of the above culture with OD₆₀₀ₙₘ=4 with 100 μL of P. simiae WCS417 culture with OD₆₀₀ₙₘ=1, carrying the T7-GFP gene. Ex-conjugants are plated on LB-agar square plates supplemented with 150 μg/mL Kna. Colonies are scraped out from plates for FACS. The constructed library is sequenced using the JGI Miseq platform.

### Fluorescence-Activated Cell Sorting (FACS)

The 2 mL culture of WCS417 strains transformed with the promoter mutant library is vortexed and then filtered through 50 μm BD filcons. Samples are loaded into a BD FACSAria II flow cytometry system (BD Biosciences, San Jose, CA) at the FACS Facility, Berkeley Lab. The nozzle size is 100 μm, laser excitation is at 488 nm, and emission is at 518 nm. Sorting is performed following the equipment's operation manual. The wild-type WCS417 strain is used as a negative control, and the WCS417 strain with the lacUV promoter w/o lacI driving the expression of the T7RNAP gene is used as a positive control. Sorted cells are plated on LB plates supplemented with 150 μg/mL Kan. To measure the GFP fluorescence of the sorted cells, emerging single colonies are inoculated in 150 μL of LB supplemented with 150 μg/mL Kan in 96-well black Corning polystyrene microplates. The plate is incubated with 800 rpm linear shaking at 28° C. for 24 h on a BioTek microplate reader. OD₆₀₀ₙₘ and GFP fluorescent signal (ex. 485/em. 528 nm) are measured at intervals of 20 min.

### Gnotobiotic Root Elongation Assay

First, Arabidopsis seeds are sterilized with 70% ethanol for 2 min and 10% bleach for 5 min. These seeds are rinsed five times with sterile water. Sterilized seeds are kept in a 4° C. fridge for 3 days and then are horizontally individually dispensed on the surface of 1/2 MS agar plates. The plates are vertically incubated in a plant growth chamber under a light cycle of 8 h dark and 16 h light for 6 days. Beginning on the evening of the fifth day, IAA and acdS strains are incubated in 3 mL of LB containing 150 mg/mL Kan overnight at 28° C. On the following day, cultures are washed three times with sterile 10 mM MgCl₂ solution and measured for values at OD₆₀₀ₙₘ using an Eppendorf Spectro reader. Uniformly germinated 6-day-old seedlings are inoculated with 1 μL of OD₆₀₀ₙₘ=0.2 culture of engineered WCS417 strains. Controls underwent mock treatment with 1 μL of 10 mM MgCl, and wild-type WCS417 strain treatment by inoculating 1 μL of OD₆₀₀ₙₘ=0.2 culture of the WCS417 strain. Treated seedlings are further incubated in a growth chamber for growth. On the seventh and fourteenth days after inoculation, images of the seedlings are captured with an Epson Perfection V850 Pro Scanner (Epson, Suwa, Nagano, Japan). The whole assay is repeated three times. Collected photos are analyzed using EZ-Root-VIS software (Shahzad et al., 2018) for primary root length, lateral root number, and total length.

For pot assay two weeks after inoculation of strains, the above seedlings are transferred into 4.9×5.25×5.25-inch pots containing soil (Hoffman 10410 Organic Cactus and Succulent Soil Mix). The pots are divided into two groups, one under the standard growth condition and the other under the drought condition (created by withholding water for more than 2 weeks.

### Microscopic Imaging

Seedlings treated with different engineered strains or with the wild-type strain are mounted on glass slides for imaging on Zeiss LSM 710 microscopes at the Advanced Microscopy Facility, Berkeley lab.

### Species Tree Construction

Annotated genomes of strains are downloaded from Integrated Microbial Genomes (webpage for: jgi.doe.gov/data-and-tools/img/). The species tree is constructed using OrthoFinder (Emms and Kelly, 2015) and the generated newick tree file is uploaded to iTOL (webpage for: itol.embl.de/) for annotation (Letunic and Bork, 2007).

### Statistical Analysis

Pairwise comparison is conducted with the independent t-test model. Three-way ANOVA is performed using the R statistical package. The statistically significant thresholds are set at 5% for one asterisk and 1% for two asterisks.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified plant-growth-promoting rhizobacterium (PGPR) to produce or increase production of an auxin or ethylene such that the PGPR is enhanced or increased in its capability to promote, enhance or increase one or more of its plant-growth-promoting (PGP) traits, property, or activity, when compared to the unmodified PGPR.

2. The genetically modified PGPR of claim 1, wherein the PGPR is increased in expression of one or more, or all, of the following enzymes: aminotransferase, IPyA decarboxylase (IPDC), and/or indole acetaldehyde dehydrogenase (IAAID), such that the PGPR produces more indole-3-acetic acid (IAA) when compared to the unmodified PGPR.

3. The genetically modified PGPR of claim 2, wherein the PGPR is increased in expression of one or more, or all, of the following enzymes: 3-deoxy-D-arabinoheptulosonate 7-phosphate (DAHP) synthase and/or anthranilate synthetase.

4. The genetically modified PGPR of claim 1, wherein the PGPR is modified to express or increase expression of 1-aminocyclopropane-1-carboxylate (ACC) deaminase, and/or decrease, or knock out, expression of ACC oxidase.

5. The genetically modified PGPR of claim 1, wherein the PGPR is a bacterium of genus Rhizobium, Agrobacterium, Ralstonia, Allorhizobium, Azorhizobium, Bradyrhizobium, Ocrhobactrum, or Pseudomonas.

6. The genetically modified PGPR of claim 5, wherein the PGPR is a bacterium of genus Pseudomonas.

7. The genetically modified PGPR of claim 6, wherein the PGPR is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, P. plecoglossicida, or P. simiae.

8. The genetically modified PGPR of claim 7, wherein the PGPR is a P. simiae.

9. A method of enhancing growth of a plant, the method comprising: (a) optionally introducing a nucleic acid encoding one or more enzyme(s) for producing an auxin or ethylene into the unmodified PGPR to generate the genetically modified PGPR of claim 1, such that the modified PGPR is capable of expressing the one or more enzyme(s), such that the genetically modified PGPR produces the auxin or ethylene; and (b) introducing or applying or contacting the genetically modified PGPR to or with a soil, wherein a plant resides, or a plant, such that the PGPR colonizes the soil so that growth of the plant is enhanced.

10. The method of claim 9, wherein the growth of the plant, or a plant part, is further enhanced at least about 10% when compared to an enhancement by an unmodified PGPR.

11. The method of claim 10, wherein the growth of the plant, or a plant part, is further enhanced at least about 20% when compared to an enhancement by an unmodified PGPR.

12. The method of claim 10, wherein the plant part is a stem, shoot or root.

13. The method of claim 9, wherein the PGPR is increased in expression of one or more, or all, of the following enzymes: aminotransferase, IPyA decarboxylase (IPDC), and/or indole acetaldehyde dehydrogenase (IAAID), such that the PGPR produces more indole-3-acetic acid (IAA) when compared to the unmodified PGPR.

14. The method of claim 12, wherein the PGPR is increased in expression of one or more, or all, of the following enzymes: 3-deoxy-D-arabinoheptulosonate 7-phosphate (DAHP) synthase and/or anthranilate synthetase.

15. The method of claim 9, wherein the PGPR is modified to express or increase expression of 1-aminocyclopropane-1-carboxylate (ACC) deaminase, and/or decrease, or knock out, expression of ACC oxidase.

16. The method of claim 9, wherein the PGPR is a bacterium of genus Rhizobium, Agrobacterium, Ralstonia, Allorhizobium, Azorhizobium, Bradyrhizobium, Ocrhobactrum, or Pseudomonas.

17. The method of claim 15, wherein the PGPR is a bacterium of genus Pseudomonas.

18. The method of claim 16, wherein the PGPR is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, P. plecoglossicida, or P. simiae.

19. The method of claim 17, wherein the PGPR is a P. simiae.
